(19) United States
(12) Patent Application Publication
(10) Pub. No.: US 2017/0021138 A1
(43) Pub. Date: Jan. 26, 2017

(54) NEEDLE ASSEMBLIES WITH PASSIVE SAFETY SYSTEMS AND RELATED METHODS

(71) Applicant: B. Braun Melsungen AG, Melsungen (DE)

(72) Inventor: Stanislav Sokolski, Greifswald (DE)

(73) Assignee: B. Braun Melsungen AG, Melsungen (DE)

(21) Appl. No.: 14/803,984

(22) Filed: Jul. 20, 2015

(51) Int. Cl. A61M 25/06

(52) U.S. Cl. CPC A61M 25/0618; A61M 2207/00

(57) ABSTRACT

Safety needle assemblies that include a needle having a sharp distal tip and a needle tip cover mounted on the needle are disclosed. The needle tip cover includes a proximal wall having an opening for the needle to pass therethrough. The needle tip cover urges against the needle in a ready to use position and shields the sharp distal tip in the secured position. The needle tip cover is moved proximally by a spring after the needle has been inserted into a patient a predetermined depth.

### FIELD OF ART

The present invention is generally directed to needle safety assemblies and related methods and more particularly to needle safety assemblies with passive safety systems and related methods.

### BACKGROUND

Insertion procedures for any needle assembly typically includes the following basic steps: (1) the healthcare worker inserts the needle into the patient's vein or tissue; (2) after insertion into the vein with the needle tip, fluid is injected into the patient or drawn from the patient by pushing or pulling a plunger attached to the needle assembly; (3) the healthcare worker withdraws the needle by grasping the needle adapter end (opposite the pointed needle tip) while at the same time applying pressure to the patient's skin at the insertion site with a cotton swab in his or her free hand to stop the flow of blood; and (4) the healthcare worker then tapes the cotton swab to the patient's skin to stop the flow of blood and cover the puncture.

One potential problem with this procedure is that immediately after the withdrawal of the needle from the patient's vein, the healthcare worker, who is at this time involved in at least two urgent procedures, must place the exposed needle tip at a nearby location and address the tasks required to accomplish the needle withdrawal. It is at this juncture that the exposed needle tip creates a danger of an accidental needle stick, which, under the circumstances, can leave the healthcare worker vulnerable to the transmission of various dangerous blood-borne pathogens, such as HIV and hepatitis.

Other needle types similarly expose healthcare workers to risks of accidental needle sticks. For example, a doctor administering an injection using a straight needle, a Huber needle, an epidural needle, etc., may place the used needle on a tray for subsequent disposal by a nurse.

For the period between placing the used needle on a tray or a work station to the time it is discarded, the used needle is a potential source for disease transmissions for those that work near or around the needle.

### SUMMARY

The various embodiments of needle assemblies have several features, no single one of which is solely responsible for their desirable attributes. Without limiting the scope of the present embodiments as set forth in the claims that follow, their more prominent features now will be discussed briefly.

A needle tip assembly provided in accordance with aspects of the present disclosure can comprise a needle adapter having an opening at a proximal end and a distal end; a needle extending distally from the distal end of the needle adapter, the needle having a needle shaft and a needle tip at a distal end of the needle shaft; a spring clip slidably riding on the needle shaft and having a proximal wall with an opening for the needle shaft to pass therethrough, the spring clip urging against the needle shaft adjacent the needle tip in a ready to use position, and shielding the needle tip in a secured position; a spring having a proximal end coupled to the needle adapter and a distal end contacting the proximal wall of the spring clip; and a securing device coupled to a latch part of the spring and compressing a portion of the spring under tension in the ready to use position and decoupled from the latch part of the spring by proximal movement of the spring clip.

The needle can further comprises a change in profile adjacent the needle tip on the needle located distal of the proximal wall of the spring clip, the opening of the spring clip has a size smaller than a size of the change in profile to prevent removing the spring clip from the needle, and the distal end of the spring is adjacent the change in profile in the secured position.

The spring clip can urge against opposite sides of the needle shaft when not in the secured position.

The needle guard can further comprise a pair of resilient arms extending distally from the proximal wall, the resilient arms urging against opposite sides of the needle shaft, and the resilient arms shielding the needle tip in the secured position.

The spring can be a compression spring.

The distal end of the spring can be attached to the proximal wall of the spring clip.

The distal end of the spring can be pressing against the proximal wall of the spring clip.

The spring clip can be freely rotatable about the spring.

The securing device can comprise an attach end coupled to the latch part of the spring to secure the spring clip in the ready to use position.

The attach end can elastically move away from the spring when the attach end is decoupled from the spring.

The latch part of the spring can be pressing against the attach end.

The attach end can be decoupled from the spring when decoupler of the spring urges proximally against the attach end.

The latch part of the spring can be a coil and the decoupler of the spring is an adjacent coil.

The attach end can be hook-shaped.

The attach end can be triangular shaped.

The securing device can comprise more than one attach end.

The securing device can further comprise a pivot point proximal of the proximal end of the spring.

The pivot point can be coupled to the needle adapter.

The securing device can further comprise a mount coupled to the needle adapter, the pivot point coupling the attach end to the mount.

The securing device can further comprise an arm having one end pivoting about the mount and another end connected to the attach end.

Almost an entire length of the spring can be under compression by the securing device.

Less than half a length of the spring can be under compression by the securing device.

The securing device can further comprise a pivot point distal of the proximal end of the spring.

The pivot point can be mounted on a different part of the spring.

The pivot point can be a loop connected to the different part of the spring.

The spring can extend nearly an entire length of the needle shaft in the ready to use position.

A further aspect of the present disclosure can include a method of making a needle tip assembly which can comprise: extending a needle distally from a distal end of a needle adapter through a spring and an opening in a proximal wall of a spring clip, the needle adapter having an opening at a proximal end, the needle having a needle shaft and a needle tip at a distal end of the needle shaft; urging the spring clip against the needle shaft adjacent the needle tip in the ready to use position; coupling a proximal end of the spring to the needle adapter; contacting a distal end of the spring with the proximal wall of the spring clip; and compressing a portion of the spring under tension with a securing device coupled to a latch part of the spring in the ready to use position, the securing device decoupling from the latch part of the spring clip for shielding the needle tip in a secured position, the securing device decoupling from the latch part of the spring by proximal movement of the spring clip.

The needle can further comprises a change in profile adjacent the needle tip on the needle located distal of the proximal wall of the spring clip, the opening of the spring clip has a size smaller than a size of the change in profile to prevent removing the spring clip from the needle, and the distal end of the spring is adjacent the change in profile in the secured position.

The spring clip can urge against opposite sides of the needle shaft when not in the secured position.

The needle guard can further comprise a pair of resilient arms extending distally from the proximal wall, the resilient arms urging against opposite sides of the needle shaft, and the resilient arms shielding the needle tip in the secured position.

The spring can be a compression spring.

The distal end of the spring can be attached to the proximal wall of the spring clip.

The distal end of the spring can be pressing against the proximal wall of the spring clip.

The spring clip can be freely rotatable about the spring.

The securing device can comprise an attach end coupled to the latch part of the spring to secure the spring clip in the ready to use position.

The attach end can elastically move away from the spring when the attach end is decoupled from the spring.

The latch part of the spring can be pressing against the attach end.

The attach end can be decoupled from the spring when decoupler of the spring urges proximally against the attach end.

The latch part of the spring can be a coil and the decoupler of the spring is an adjacent coil.

The attach end can be hook-shaped.

The attach end can be triangular shaped.

The securing device can comprise more than one attach end.

The securing device can further comprise a pivot point proximal of the proximal end of the spring.

The pivot point can be coupled to the needle adapter.

The securing device can further comprise a mount coupled to the needle adapter, the pivot point coupling the attach end to the mount.

The securing device can further comprise an arm having one end pivoting about the mount and another end connected to the attach end.

Almost an entire length of the spring can be under compression by the securing device.

Less than half a length of the spring can be under compression by the securing device.

The securing device can further comprise a pivot point distal of the proximal end of the spring.

The pivot point can be mounted on a different part of the spring.

The pivot point can be a loop connected to the different part of the spring.

The spring can extend nearly an entire length of the needle shaft in the ready to use position.

Aspects of the present disclosure can also include a safety needle assembly which can comprise a needle attached to a needle adapter, a spring mounted on the needle with a tip protector, and a securing mechanism for maintaining at least a portion the spring in a compressed state in a ready to use position.

### BRIEF DESCRIPTION OF THE DRAWINGS

The various embodiments of the present safety needle assemblies now will be discussed in detail with an emphasis on highlighting the advantageous features. These embodiments depict the novel and non-obvious safety needle assemblies shown in the accompanying drawings, which are for illustrative purposes only. These drawings include the following figures, in which like numerals indicate like parts:

FIGS. 1A-1C depict an embodiment of a safety needle assembly with a passive safety system prior, during, and after activation; and

FIGS. 2A-2C depict another embodiment of a safety needle assembly with a passive safety system prior, during, and after activation.

FIGS. 3A-3C depict yet another embodiment of a safety needle assembly with a passive safety system prior, during, and after activation.

### DETAILED DESCRIPTION

The following detailed description describes the present devices, apparatuses, systems, and methods with reference to the drawings. In the drawings, reference numbers label elements of the present embodiments. These reference numbers are reproduced below in connection with the discussion of the corresponding drawing features.

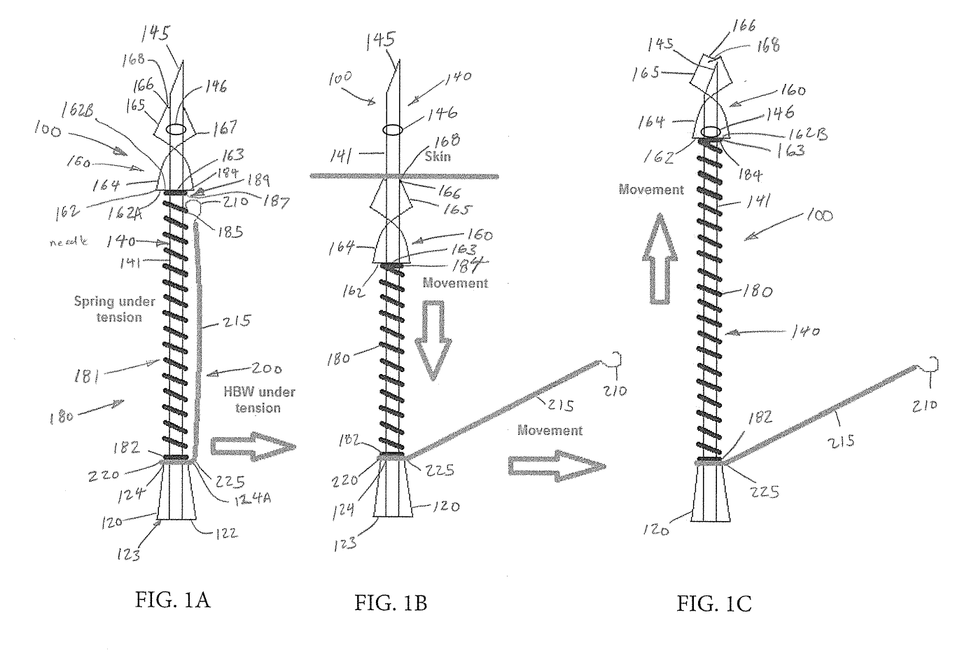

Figures (“FIGs.”) 1A-1C illustrate an embodiment of a safety needle attachment, device, or assembly 100 provided in accordance with aspects of the present disclosure shown at various stages of operation. FIG. 1A illustrates an initial state of the needle assembly 100 in a ready to use position in which the needle tip of the needle is exposed, FIG. 1B shows the needle assembly during use, such as for performing an injection, and FIG. 1C shows the needle assembly after use in a protective position, such as being protected by a safety system.

Referring now to FIG. 1A, the safety needle assembly 100 comprises a needle adapter 120, a needle 140, a needle guard or spring clip 160 slidably carried on the needle 140, a spring 180 positioned between the needle adapter 120 and the spring clip 160, and a securing device 200 compressing a portion of the spring 180 in a ready to use position. The needle adapter 120 has a proximal end 122 and a distal end 124 and may alternatively be referred to as a hub. The needle 140 projects distally from the distal end 124 of the needle adapter 120. The proximal end 122 has an opening 123 for receiving a male medical implement such as a syringe for injecting fluid into the tissue of the patient or drawing fluid or blood from the patient. In some examples, the opening 123 is a female Luer for receiving a male Luer tip, such as a syringe tip. In other examples, the exterior of the needle adapter 120 has exterior threads for threaded engagement with a threaded collar on the syringe or other medical implements.

A ledge 124A is provided at the interface of the needle 140 and the distal end 124 of the needle adapter 120. The overall shape of the needle adapter 120 may vary and therefore vary the size and shape of the ledge 124A. In some examples, no material circular ledge is formed 124A and the distal end of the needle adapter 120 is approximately the size of the needle shaft. As shown, an outer surface of the needle adapter 120 tapers inward from the proximal end 122 to the distal end 124 to form a frustoconical shaped grip for a user to handily attach the safety needle assembly 100 to a syringe or other medical devices. A distal end of the frustoconical shaped grip has a ring shaped planar surface defining the ledge 124A for supporting a safety feature, as further discussed below. In some examples, a cross-section of the needle adapter 120 can be circular, rectangular, or any regular or irregular shape to form the grip. Surface features, such as fins and bumps, may be added to form a more effective gripping surface.

The needle 140 comprises a needle shaft 141 having a proximal end attached to the needle adapter 120, a needle tip 145 formed at a distal end of the needle shaft 141, and a change in profile 146 positioned adjacent the needle tip 145 on the needle shaft 141. The proximal end of the needle shaft 141 can be press fitted into the distal end 124 of the needle adapter 120 or glued into a glue well at the distal end 124 of the needle adapter 120. The needle tip 145 is configured to puncture the skin and tissue of a patient so that fluid can be injected into the patient, such as from a syringe attached to the needle adapter 120, or blood or other bodily fluid can be withdrawn through the needle tip 145 into the syringe. The needle tip 145 can be cut or ground to have any known prior art tip geometries, including a non-coring needle tip. The change in profile 146 has a cross-section larger than a cross-section of the needle shaft 141 to prevent the spring clip 160 from detaching from the safety needle assembly 100, as will be discussed below in reference to FIG. 1C. The change in profile 146 can be a crimp, a bulge, a sleeve, an attachment, or a material buildup.

A spring 180 is provided with the needle 140 to act as a passive safety device configured to move the spring clip or tip protector 160 in a distal direction to protect the tip from unintended needle sticks following use. In the illustrated embodiment, the spring 180 is a compression spring having a plurality of interconnected coils between the proximal end 182 and the distal end 184 of the spring. The proximal end 182 of the spring 180 can be placed in abutting contact with the ledge 124a of the needle adapter 120. For example, the proximal end most coil of the spring 180 can be placed in abutting contact with the generally planar surface of the ledge 124a. To prevent the spring from separating from the needle adaptor 120, the spring can be secured to the needle adaptor, such as by mechanical inter-engagement, glue, bonding, or other securement means. In some examples, the proximal end 182 of the spring 180 can be attached directly to the needle shaft 141 at or near the interface of the needle 140 and the needle adapter 120. The distal end 184 of the spring 180 is attached to or pressed against the spring clip 160, or more specifically, to a proximally facing surface 162A of a proximal end wall 162 of the spring clip 160 to propel the spring clip 160 distally towards the needle tip 145 to shield the needle tip from inadvertent needle sticks. In one example, the distal end 184 of the spring 180 presses against or abuts the spring clip 160 so that the spring clip 160 can freely rotate about the needle shaft 141 relative to the spring 180. In another example, the distal end 184 is attached to the proximally facing surface of the proximal wall 162 so that the spring clip 160 is not free to rotate about the needle shaft 141 relative to the spring 180. As shown in the figures, the spring 180 extends along nearly the entire length of the needle shaft 141 from the ledge 124a of the needle adapter 120.

Characteristics of the spring 180, such as the spring material, coil diameter, wire diameter or thickness, and pitch can be selected depending on the particular application of the safety needle assembly 100. For example, if the needle 140 is to extend deep into the patient, a low spring constant can reduce discomfort of the spring clip 160 pressing against the patient. A minimum spring constant may be achieved if the spring 180 can extend nearly an entire length of the exposed needle shaft 141 when the needle tip 145 is secured by the spring clip 160, as shown in FIG. 1C. That is, the longer the length of the spring 180 along the needle 140, the lower the spring constant can be achieved.

The spring 180 has a latch point 185 located between the proximal end 182 and the distal end 184. The latch point 185 can be a portion or a point of a coil for use by a securing device 200 to engage or latch onto. In some examples, the latch point 185 can be a tab, a hook, a protrusion, or an attachment extending outwardly from a coil of the spring 180 for the securing device 200 to latch onto. When the securing device 200 engages the latch point 185 of the spring 180, the spring 180 is divided between an uncompressed portion 189 distal of the latch point 185 and a compressed portion 181 proximal of the latch point 185. In some examples, the securing device 200 engages two latch points 185 on the coil along two spaced positions on the coil. The uncompressed portion 189 of the spring is defined between the latch point 185 and the distal end 187 of the spring, which can be contacted or abutted by a proximal wall of the spring clip 160. The compressed portion 181 of the spring is defined between the proximal end 182 and the latch point 185. As shown, nearly the entire length of the spring 180 is under compression or, put in another way, the uncompressed portion is zero or close to zero. In another example, at least 80% of the entire uncompressed length of the spring is under compression or, put in another way, the uncompressed portion is less than 20% of the entire length of the spring 180 between the spring clip 160 and the proximal end 182. In yet another example, at least 50% of the entire length of the spring 180 is under compression or, put in another way, the uncompressed portion of the spring is less than 50% of the entire length between the spring clip 160 and the proximal end 182.

The securing device 200, which may also be referred to as a hold back wire (HBW), can comprise a mount 220 disposed between the proximal end 182 of the spring 180 and the ledge 124a, an attach end 210 configured to engage with the latch point 185, and a securing arm 215 coupling the attach end 210 to the mount 220. In some examples, the mount 220 can be attached directly to the needle adapter 120 at a side of the ledge 124a so that the proximal end 182 of the spring 180 directly contacts the ledge 124a. As shown, the mount 220 has an opening for the needle 140 to extend therethrough and is pressed against the ledge 124a by the spring 180. In some examples, the mount 220 is pivotably connected to a coil of the spring 180 at or near the proximal end 182 of the spring 180.

The securing arm 215 can be pivotably attached to the mount 220 via a pivot point 225 as shown. The mount 220 can embody a coiled loop and unitarily formed with the securing arm 215 and the pivot point 225 can be a bend. In some examples, the securing arm 215 can be pivotably mounted directly to the needle adapter 120 without the need for a mount 220, such as being anchored directly into the wall surface of the needle adapter. The securing arm 215 normally extends at least partially radially outward from the needle 180 so that when the securing arm 215 is pivoted about the pivot point 225 and the attach end 210 engages the latch point 185, elastic potential energy is stored in the securing arm 215 and the pivot point 225. That is, in the normal state, the securing arm 215 and the attach end 210 at the free end of the securing arm are pointed away from the spring 180 and the needle 140. When the attach end 210 is engaged with the latch point 185 in the initial position, the securing arm 215 is simultaneously under tension caused by compressing the compressed portion 181 of the spring 180 and elastically deflected inwardly towards the spring 180 and the needle 140 caused by pivoting or bending the securing arm 215 towards the spring 180 to engage the attach end 210 with the latch point 185. Elastic potential energy is stored in the securing arm 215 and the pivot point 225 from the elastic deflection. The tension in the securing arm 215 caused by the spring 180 under compression maintains the engagement between the attach end 210 and the latch point 185 and prevents the securing arm 215 and the attach end 210 from elastically returning outwardly away from the spring 180.

In some examples, the attach end 210 can comprise a hook, a lever, a lip, a notch, a shoulder, a spline, or combinations thereof. The attach end 210 can removably engage the latch point 185 and upon compression of the spring 180 separates and deflects radially outwardly from the latch point 185, as further discussed below. The removable engagement can be a simple overlap with friction, a positive engagement with part of the attach end 210 entering part of the latch point 185 or combinations thereof.

The securing device 200 is coupled to the latch point 185 via the attach end 210 thereby dividing the spring 180 into the compressed portion 181 proximal of the latch point 185 and the uncompressed portion 189 distal of the latch point 185 in the ready to use position, as previously discussed. The location of the latch point 185 between the proximal and distal ends of the spring 180 can determine how far the spring clip 160 can travel in the proximal direction, such as when pressed against and pushed by the skin in the proximal direction during an injection, before the attach end 210 decouples from the latch point 185. As shown, the latch point 185 is located near a distal end 184 of the spring so a relatively small displacement of the spring clip 160 in the proximal direction can decouple the attach end 210 from the spring 180. In another example, if the latch point 185 is located near a proximal end 182 of the spring 180, then a greater displacement of the spring clip 160 is required before the attach end 210 is decoupled from the spring 180. Therefore, depending on the penetration depth of the needle 140 into the patient, the location of the latch point 185 and attach end 210 along the length of the spring 180 can be selected by the required proximal movement of the spring clip 160.

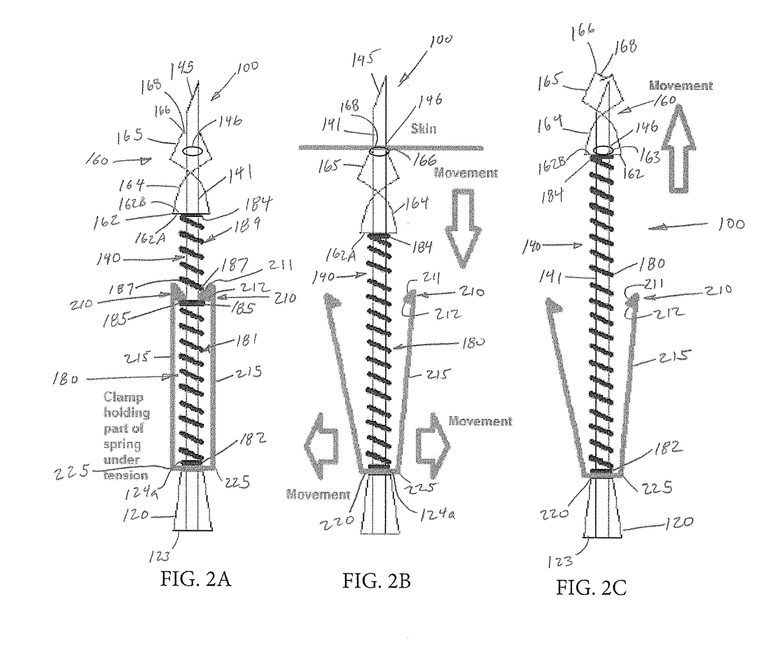

The attach end 210 is decoupled from the latch point 185 of the spring 180 by movement of the latch point 185 relative to the attach end 210 until contact between the latch point 185 and the attach end 210 is removed. In examples where a simple friction engagement is utilized, such as the embodiment of FIGS. 2A-2C, the attach end 210 can be moved relative to the latch point 185 to decouple the attach end from the latch point. In an example, a decoupler 187 can be incorporated to decouple the latch point 185 from the attach end 210. The decoupler 187 can press against the attach end 210 and exert at least a radially outwardly force or component forces to separate the contact with the latch point 185. As shown in FIGS. 1A and 1B, the decoupler 187 can be an adjacent coil immediately distal of the latch point 185. Thus, the decoupler 187 can either contact and deflect the attach end 210 radially outwardly, can compress the coils of the spring to move the latch point 185 in a proximal direction away from the attach end 210, or both to separate the latch point 185 and the attach end 210 from one another. In some examples, the decoupler 187 can be an attachment or protrusion extending from the adjacent coil distal of the latch point 185. In other examples, the decoupler can be the spring clip 160 and the spring clip exerts a downwardly force or component forces to separate the latch point 185 and the attach end 210 from one another.

In an example, the decoupler 187 operates by contacting and pressing the attach end 210 outwardly from the latch point 185 until the attach end 210 is no longer engaged with the latch point 185. In some examples, the decoupler 187 presses against the latch point 185 and moves the latch point 185 proximally until the attach end 210 no longer engages the latch point 185. As shown in FIGS. 1A-1C, the attach end 210 can be shaped like a hook with the free end of the hook engaging with the latch point 185. Because the compressed portion 181 of the spring is held in a compressed state by the attach end 210 of the securing device 200, the safety needle assembly 100 is in an equilibrium state in the ready to use position of FIG. 1A.

With reference to FIG. 1B, the decoupler 187 is moved proximally when the needle 180 is inserted into the patient thereby moving the spring clip 160 proximally towards the latch point 185. As the spring clip 160 advances proximally on the needle, the uncompressed portion 189 starts to compress and the decoupler 187 advances towards the attach end 210. Further proximal movement causes the decoupler 187 to press against the attach end 210 and forces the attach end 210 to decouple or disengage from the latch point 185. Alternatively or in combination therewith, further proximal movement causes the compressed portion 181 of the spring to further compress, which moves the latch point 185 proximally away from the attach end 210. The securing arm 215 then elastically springs outwardly away from the spring 180 due to the stored elastic potential energy and remains out of the path of the spring 180 as shown in FIGS. 1B and 1C. Once the attach end 210 is no longer engaged with the latch point 185, the spring clip 160 can further move proximally depending on the depth of the needle insertion and the compressed spring 180 is ready to expand upon removal of the needle from the patient to move the tip protector or spring clip 160 over the needle tip, as shown in FIGS. 1B and 1C.

The spring clip 160 is prevented from moving distally by the securing device 200 compressing the compressed portion 181 of the spring 180 in the ready to use position. The spring clip 160 is configured to slide along the needle shaft 141 to shield the needle tip 145 from unintended needle pricks following activation, such as following use and the attach end is separated from the latch point. The spring clip 160 can slide proximally from the initial or ready to use position shown in FIG. 1A to an in-use position shown in FIG. 1B where the spring clip is further spaced from the needle tip, and then distally to the after-use, closed, or secured position shown in FIG. 1C where the spring clip is effectively located over the needle tip to shield the needle tip, such as after the needle 140 is removed from the patient.

As shown, one embodiment of the spring clip 160 of the present disclosure comprises a proximal wall 162 and two resilient arms 164 extending distally from the proximal wall 162. In one example, the spring clip 160 has a proximal wall 162 having a proximally facing surface 162A and a distally facing surface 162B. The proximal wall 162 can be generally circular, round or rectangular and can include a circular or non-circular aperture or perimeter defining an opening 163 for the needle 140 to extend therethrough. The opening 163 can be sized larger than the needle shaft diameter but smaller than a size of the change in profile 146 to prevent the proximal wall 162 from moving distally past the change in profile 146 in the secured position shown in FIG. 1C. In other examples, the spring clip 160 can embody a number of different guard devices or configurations for covering and securing the needle tip 145 from inadvertent needle sticks after withdrawing the safety needle following use. Exemplary spring clips 160 can be found in U.S. Pat. Nos. 6,616,630 and 8,827,965 and as the needle safety element presented in U.S. patent application Ser. No. 13/257,572, published as US 2012/0046620 A1, the contents of which are expressly incorporated herein by reference.

In the illustrated embodiment, each resilient arm 164 comprises a distal wall 165 having a free end 166 biased against diametrically opposite portions or sides of the needle shaft 141 when not in the secured position. As shown, the resilient arm 164 also has an elbow 167 extending from the wall 162 and a tip 168 at the free end 166 of the arm 164. The tip 168 can be a curved lip or hook which allows the free ends 166 to slide smoothly along the needle shaft 141. In some examples, the tips 168 can each have a simple straight edge with optional low friction material added to improve sliding relative to the surface of the needle, such as medical grade lubricant. The free ends 166 of the spring clip 160 are slidably positioned on opposite sides of the needle shaft 141 to allow the spring clip 160 to slide axially while minimizing any moment about the needle 140. As shown, the tips 168 of the two resilient arms 164 press against the needle shaft 141 in the ready to use position of FIG. 1A and the in-use position shown in FIG. 1B. The resilient arms 164 are elastically deflected outwardly by the needle shaft 141 and the ends 166 of the distal walls 165 are pressed against diametrically opposite points of the needle shaft 141.

Referring now to FIG. 1B, when the needle 140 is inserted into the patient, the spring clip 160 is moved proximally along the needle shaft 141 by abutting against the skin of the patient.

As the needle 140 is inserted deeper into the patient, the spring clip 160 moves proximally thereby compressing the uncompressed portion 189 of the spring 180 and bringing the decoupler 187 closer to the latch point 185. Eventually, the decoupler 187 of the spring 180 presses against the attach end 210 or moves the latch point 185 proximally of the attach end 210 to disengage the attach end 210 from the latch point 185. Once the attach end 210 is no longer engaged with the latch point 185, the elastic securing arm 215 elastically springs outwardly away from the spring 180 and remains displaced from the spring 180. At this point, the spring clip 160 is pressed against the skin with the full length of the spring 180 unrestricted by the securing device 200.

Referring now to FIG. 1C, as the needle is 140 is removed from the patient, the spring clip 160 slides distally along the needle shaft 141 by the expansion force of the spring until the proximal wall 162, and more specifically the perimeter defining the opening 163 on the proximal wall, contacts the change in profile 146 on the needle, which stops further distal movement of the spring clip and further expansion of the spring. Before, concurrently, or shortly thereafter, the resilient arms 164 of the spring clip 160, now no longer pressing against the needle shaft 141, activate and move radially to move the two distal walls over the needle tip 145 to cover the needle tip 145 in the secured position. In the secured position, the free ends 166 or the tips 168 of the arms 164 slide off the needle shaft and elastically move towards each other to cover the needle tip 145 to prevent inadvertent needle sticks. In some examples, the distal walls can each have folded tabs, such as two or more folded tabs, to define a holding space for capturing the needle tip therein to prevent the needle tip from moving laterally outside the side edges of the arms 164. The arms 164 can also have different lengths so that when the spring clip 160 is activated, the two distal walls 165 are axially offset and covering the needle tip.

The engagement between the change in profile 146 and the opening 163 in the proximal wall 162 of the spring clip 160 prevents spring clip 160 from displacing distally off of the needle. Specifically, the distally facing surface 162B, such as the perimeter defining the opening 163, of the proximal wall 162 abuts against the change in profile 146 to prevent further distal movement of the spring clip following movement to the used or protected position in which the needle tip is shielded. Because the change in profile 146 is larger in size than the opening 163, the spring clip 160 cannot slide past the change in profile, thereby confining the needle tip 145 within the spring clip 160 and preventing unintended needle sticks after the needle 140 has been removed from the patient.

In some examples, a cylinder or housing having a cavity for accommodating the spring clip 160 is contemplated. For example, the housing can resemble a cup having an elongated wall, one close end with an opening for the needle and one open end, which can be larger in size than the opening on the closed end. The housing can be placed at a distal end of the spring or somewhere near the distal end. The housing can be sized and shaped to accept the spring clip therein and allow the arms of the spring clip to move between the ready to use position and the protective position, shown in FIG. 1C. The housing can have a distal end edge that extends distal of the spring clip 160 so that when the needle is used on a patient, the skin pushes against the distal end edge of the housing and not the spring clip 160. In some examples, the elongated wall can be a partially continuous, such as being semi-enclosed or having only part of a circumference. This housing can be practiced with any of the needle assemblies discussed elsewhere herein.

FIGS. 2A-2C illustrate another embodiment of a safety needle assembly 100 provided in accordance with further aspects of the present disclosure. The present safety needle assembly 100 is similar to the safety needle assembly of FIGS. 1A-1C with a few exceptions. In the present embodiment, the securing device 200 comprises two attach ends 210 and the configuration of the attach ends 210 is also different. Each attach end 210 of the present embodiment has an engaging surface 212 and a pressing surface 211 angled from the engaging surface 212. The securing arm 215 extends from the engaging surface 212 of each attach end 210 and can be pivotally attached to the mount 220 or the needle adapter 120 at the pivot point 225. As shown, the mount 220 is sandwiched between the proximal end 182 of the spring 180 and the ledge 124a of the needle adapter 120.

With reference now to FIG. 2A, each attach end 210 is engaged with a latch point 185 located on opposite sides of the spring 180 to secure the needle assembly in a ready to use position. In this position, the spring 180 has a compressed portion 181 between the latch points 185 and the proximal end 182 of the spring 180 and an uncompressed portion 185 between the latch point 185 and the spring clip 160. The position of the latch points 185 for the two attach ends 210 along the length of the spring are shown closer to the distal end 184 of the spring 180 than the proximal end 182 of the spring 180, but can be positioned anywhere along the length of the spring 180 depending on, as an example, the depth of needle insertion into the patient. Thus, the position of the latch points relative to the length of the spring is selectable depending on the desired needle application. In some examples, the latch points 185 can be axially offset to match with axially offset attach ends 210.

Decouplers 187 are incorporated and are located on an adjacent coil distal of the coil with the latch points 185. In one example, the decouplers are located on the same distal coil. In another example, the decouplers are located on different coils. In some embodiments, a multi-stage release system is contemplated with the latch points 185 staggered along the length of the spring 180 such that the securing arms 215 have unequal lengths to form a first and second compressed portions and one uncompressed portion. For example, a first latch point can be located closer to the distal end 184 of the spring 180 and a second latch point can be located between the first latch point and the proximal end 182 of the spring 180. In this example, the first compressed portion is defined between the first latch point and the second latch point, and the second compressed portion is located between the second latch point and the proximal end of the spring. In the first stage, the first decoupler disengages the first attach end from the first latch point so that the first compressed portion and the uncompressed portion combine to form a new uncompressed portion. In the second stage, the second decoupler disengages the second attach end from the second latch point to fully release the spring 180 from the securing mechanism 200.

The shape of each of the attach ends 210 can also affect the required displacement of the decoupler, the spring clip, the housing for accommodating the spring clip, or all the above before separation between the release ends and the attach points. The shape and size of the attach end 210 can determine how far the decoupler 187 must travel before abutting and pushing the attach end and decoupling the attach end 210 from the latch point 185 of the spring 180. For example, if the pressing surface 211 is closer to the decoupler 187 of the spring 180 or had a different angle in the ready to use position as shown in FIG. 2A, then a smaller amount of movement of the spring clip 160 may release the attached points to free the compressed portion 181 of the spring 180 than if the pressing surface 211 is further proximally away from the decoupler 187. In some examples, the needle device can be practiced with a single attach end 210 for attaching to a single latch point. In other examples, there can be three or more attached ends for attaching to three or more attach points.

Referring now to FIG. 2B, when the needle 180 is inserted into the patient, the spring clip 160 is moved proximally towards the two latch points 185. As the spring clip 160 moves proximally towards the latch points, the decouplers 187 located on an adjacent coil distal of the latch points 185 strike the pressing surfaces 211 of the attach ends 210. Because the pressing surface 211 is angled with respect to the engaging surface 212, the proximal movement of the decoupler 187 pushes the attach end 210 outwardly away from the spring 180, due to component forces, until the engaging surface 212 is no longer engaging the latch point 185. At this point, the two securing arms 215 and the two attach end 210 elastically spring outwardly away from the spring 180 and release the compressed portion 181 of the spring 180. In some examples, two decouplers 187 are axially offset or staggered so that one pair of securing arm 215 and attach end 210 will release prior to the release of the second pair of securing arm 215 and attach end 210. Furthermore, if the latch points 185 are staggered or axially space, the first compressed portion on one side of the spring is released when the first decoupler disengages the first attach end from the first latch point and the second compressed portion on the opposite side of the spring is released when the second decoupler disengages the second attach end from the second latch point.

With reference now to FIG. 2C, as the needle 140 is withdrawn from the patient, the spring clip 160 is advanced distally by the spring 180 towards the needle tip 145 until the needle tip is covered by the spring clip 160. Distal movement of the spring clip 160 is stopped by the proximal wall engaging the change in profile near the needle tip, as previously discussed with reference to the first embodiment.

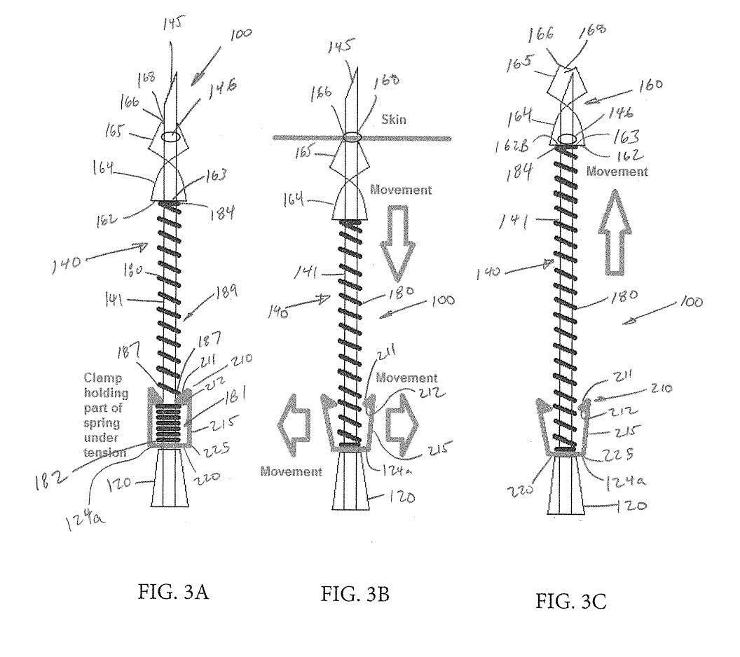

With reference now to FIGS. 3A-3C, the illustrated embodiment of the safety needle assembly 100 is similar to the safety needle assembly of FIGS. 2A-2C except that the attach ends 210 and the latch points 185 are located closer to the proximal end 182 of the spring 180 compared to the embodiment of FIGS. 2A-2C. In one example, the attach ends 210 can compress about half the length of the spring 180. In another example, the attach ends 210 can compress less than half the length of the spring 180. The longer the uncompressed portion 189 than the compressed portion 181, the longer the spring clip 160 can be displaced before the decouplers 187 disengage the attach ends 210 from the latch points 185.

Referring to FIG. 3A, the longer uncompressed portion 189 allows the spring clip 160 to travel proximally a longer distance before the attach end 210 releases the compressed portion 181 of the spring 180. This allows the needle 140 to penetrate deeper into the patient before the compressed portion 181 of the spring 180 is released.

Referring to FIG. 3B, when the needle 140 is inserted into the patient, the spring clip 160 moves proximally thereby compressing the uncompressed portion 189 of the spring 180. In the illustrated embodiment, because the uncompressed portion 189 is longer than the compressed portion 181, the amount of force required to move the spring clip 160 proximally is less than if the uncompressed portion 189 was shorter, assuming other spring characteristics being the same. The longer the uncompressed portion 189, the less the pressure applies by the spring clip 160 against the patient. Other factors, such as the spring constant of the spring 180 and the choice of material of the spring 180 can also affect the overall pressure of the spring clip 160 against the patient when inserting the needle into the patient. As the spring clip 160 is moved proximally towards the needle adapter 120 by pressing against the skin of the patient, the decoupler 187 pushes against the pressing surfaces 211 of the two attach ends 210 thereby forcing the attach ends 210 down and away from the latch points 185 of the spring 180 until both attach ends 210 are decoupled from the latch points 185 of the spring 180. As each attach end 210 is decoupled from the part of the spring 180, the flexible arm 215 springs outwardly away from the spring 180.

Referring to FIG. 3C, as the needle 140 is removed from the patient, the spring clip 160 moves distally under spring force towards the needle tip 145 while providing resistance against the patient with decreasing force, which is typical of spring force characteristics of an expansion spring. When the needle 140 is completely removed from the patient, the ends 166 of the spring clip 160 move distal of the needle tip 145 and elastically spring back towards each other to cover the needle tip 145. The change in profile 146 prevents the spring clip 160 from further distal movement to prevent removing the spring clip 160 from the needle 140.

Methods of making and of using the needle assemblies and their components described elsewhere herein are contemplated and are considered within the scope of the present disclosure.

The above description presents various embodiments of the present invention, and the manner and process of making and using them, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention.

What is claimed is:

1. A safety needle assembly, comprising:
a needle adapter having an opening at a proximal end and a distal end;
a needle extending distally from the distal end of the needle adapter, the needle having a needle shaft and a needle tip at a distal end of the needle shaft;
a spring clip slidably riding on the needle shaft and having a proximal wall with an opening for the needle shaft to pass therethrough, the spring clip urging against the needle shaft adjacent the needle tip in a ready to use position, and shielding the needle tip in a secured position;
a spring having a proximal end coupled to the needle adapter and a distal end contacting the proximal wall of the spring clip; and
a securing device under tension in the ready to use position coupled to a latch part of the spring and compressing a portion of the spring, said securing device decoupled from the latch part of the spring when the spring clip moves in a proximal direction.

2. The safety needle assembly of claim 1, wherein the needle further comprises a change in profile adjacent the needle tip on the needle located distal of the proximal wall of the spring clip, the opening of the spring clip has a size smaller than a size of the change in profile to prevent removing the spring clip from the needle, and the distal end of the spring is adjacent the change in profile in the secured position.

3. The safety needle assembly of claim 2, wherein the spring clip is urging against opposite sides of the needle shaft when not in the secured position.

4. The safety needle assembly of claim 3, wherein the needle guard further comprises a pair of resilient arms extending distally from the proximal wall, the resilient arms urging against opposite sides of the needle shaft, and the resilient arms shielding the needle tip in the secured position.

5. The safety needle assembly of claim 1, wherein the securing device comprises an attach end coupled to the latch part of the spring to secure the spring clip in the ready to use position, wherein the attach end elastically moves away from the spring when the attach end is decoupled from the spring.

6. The safety needle assembly of claim 5, wherein the attach end is decoupled from the spring when the latch part moves proximally away from the attach end.

7. The safety needle assembly of claim 5, wherein the attach end is decoupled from the spring when a decoupler of the spring urges proximally against the attach end.

8. The safety needle assembly of claim 6, wherein the latch part of the spring is a coil and the decoupler of the spring is an adjacent coil.

9. The safety needle assembly of claim 5, wherein the securing device comprises more than one attach end.

10. The safety needle assembly of claim 1, wherein the spring extends nearly an entire length of the needle shaft in the ready to use position.

11. A method of making the safety needle assembly, comprising:
extending a needle distally from a distal end of a needle adapter through a spring and an opening in a proximal wall of a spring clip, the needle adapter having an opening at a proximal end, the needle having a needle shaft and a needle tip at a distal end of the needle shaft;
urging the spring clip against the needle shaft adjacent the needle tip in the ready to use position;
coupling a proximal end of the spring to the needle adapter;
contacting a distal end of the spring with the proximal wall of the spring clip; and
compressing a portion of the spring with a securing device coupled to a latch part of the spring in the ready to use position, the securing device decouples from the latch part to release the spring clip to shield the needle tip in a secured position, and wherein the securing device decouples from the latch part by proximal movement of the spring clip.

12. The method of claim 11, wherein the needle further comprises a change in profile adjacent the needle tip on the needle located distal of the proximal wall of the spring clip, the opening of the spring clip has a size smaller than a size of the change in profile to prevent removing the spring clip from the needle, and the distal end of the spring is adjacent the change in profile in the secured position.

13. The method of claim 12, wherein the spring clip is urging against opposite sides of the needle shaft when not in the secured position.

14. The safety needle assembly of claim 13, wherein the needle guard further comprises a pair of resilient arms extending distally from the proximal wall, the resilient arms urging against opposite sides of the needle shaft, and the resilient arms shielding the needle tip in the secured position.

15. The method of claim 11, wherein the securing device comprises an attach end coupled to the latch part of the spring to secure the spring clip in the ready to use position, wherein the attach end elastically moves away from the spring when the attach end is decoupled from the spring.

16. The method of claim 15, wherein the attach end is decoupled from the spring when a decoupler urges proximally against the attach end.

17. The method of claim 16, wherein the latch part of the spring is a coil and the decoupler of the spring is an adjacent coil.

18. The method of claim 15, wherein the securing device comprises more than one attach end.

19. The method of claim 11, wherein the spring extends nearly an entire length of the needle shaft in the ready to use position.

20. A safety needle assembly comprising a needle attached to a needle adapter, a spring mounted on the needle with a tip protector, and a securing mechanism for maintaining at least a portion the spring in a compressed state in a ready to use position.
